(19) United States
(12) Patent Application Publication
(10) Pub. No.: US 2024/0123110 A1
(43) Pub. Date: Apr. 18, 2024

(54) Formulations, Biodegradable Membranes, Products Including Biodegradable Membranes, and Methods of Manufacture

(71) Applicant: TISANE LIMITED, Hamilton (NZ)

(72) Inventors: Robert Thorne LAWRENCE, Hamilton (NZ); Joy Chiung-Yi Tseng LAWRENCE, Hamilton (NZ)

(21) Appl. No.: 18/269,083

(22) PCT Filed: Dec. 23, 2021
(86) PCT No.: PCT/NZ2021/050231

§ 371 Date: Jun. 22, 2023

(30) Foreign Application Priority Data
Dec. 23, 2020 (NZ) 771399
Mar. 19, 2021 (AU) 2021900812

(51) Int. Cl. A61L 15/34; A61L 15/28; A61L 15/40

(52) U.S. Cl. CPC A61L 15/34; A61L 15/28; A61L 15/40

(57) ABSTRACT

The specification describes membranes, formulations, methods of manufacture, and methods to develop formulations and membranes that have desired physical properties. The membranes and formulations are biodegradable, in conditions like domestic and commercial compost systems. The formulations and membranes may also be flexible and have desired physical properties such as melting point and flexibility.

### STATEMENT OF CORRESPONDING APPLICATIONS

The present application is based on New Zealand Patent Application No. 771339 filed on 23 Dec. 2020 and Australian Patent Application No. 2021900812 filed on 19 Mar. 2021, the entire contents of which are incorporated herein by reference.

### 1. FIELD OF INVENTION

The present invention relates to formulations and sealant formulations, biodegradable membranes, their methods of manufacture and products incorporating the biodegradable membranes. In some forms the biodegradable membranes may be used to manufacture products such as sanitary products.

### 2. BACKGROUND TO THE INVENTION

Plastics are synthetic or semi-synthetic materials that use polymers as a key building block. These materials have properties that make them suitable for use in a wide range of applications and industries. For instance, many plastics are fluid proof, flexible, easily manufacturable into different shapes or products, non-reactive and food safe.

As a result of these properties, plastics have become ubiquitous in modern society and industry. For example, plastic materials and products are used in the building industry, food and beverage packaging, fast moving consumer goods, the automotive industry, medical products and sanitary items such menstrual pads or diapers.

However, the properties that make plastics suitable for a wide range of applications contribute to them causing significant environmental damage. For instance, plastics are inherently slow to decompose in natural ecosystems, and the conditions experienced in at-home or commercial composting systems. Often, currently available plastics, including those that are promoted as being “compostable” or “home-compostable” leave micro-plastic residues. This is a particularly worry example of “green-washing” in product marketing.

In addition, many products or packages made from plastics are intended as single-use items, meaning that they either create significant landfill or contribute to pollution if improperly disposed of by consumers.

The problems of single-use plastics materials are ubiquitous to modern life. Examples of single use products include plastic wrapping for pallets and building materials, garbage bags, shopping bags, food packaging such as is used in delicatessens, through to planter bags for plants. In the interests of brevity, the problems with single use plastics are exemplified herein with reference to one example class of product—being sanitary products. However, this is not a limitation on the scope of the present technology.

Disposable, single-use sanitary items are a common, and widely used to absorb and contain released body fluids. During menstruation, adult and infant incontinence episodes, surgery and animal transportation these allow convenient hygienic collection and handling of body fluids. These items allow full participation in education, employment and recreational activities.

However, sanitary products are not limited to only menstrual products. Other examples include (but are not limited to) diapers (nappies) for infants, incontinence products, medical underlays and dressing, veterinary underlays, pet urinary training pads and other items which absorb and retain fluids.

These products frequently comprise an absorbent layer of material and a plastic fluid-proof coatings or carrier.

Disposable single-use sanitary products are economically and societally undesirable. One estimate is that sanitary products create 4.5 million tonnes of waste which goes to landfill globally annually. There are severe environmental consequences arising from disposal of these products due to slow or non-degradation of these products and/or the toxic contaminants produced during degradation, causing undesirable hazards for the environment and climate, while also reducing land resources. These factors combined create cost, societal and environmental threats which manufacturers, consumers and governments are seeking to mitigate and reduce.

Re-usable menstrual management products are available and are becoming more widespread. However, many people have concerns with those products including aesthetic, hygiene and convenience issues. Those issues are a deterrent or barrier to people reducing the waste created by use of sanitary products. There would be strong resistance from women worldwide against any attempt to limit their access to types of sanitary management enabling broader life-opportunities without creating more waste.

### 3. OBJECT OF THE INVENTION

It may be an object of the invention to provide one or more of a membrane, a formulation, and a product which includes a formulation, which is compostable.

It may be an object of the invention to provide a membrane, a product which includes a membrane, a formulation, and methods of manufacturing a membrane and formulation.

It may be an object of the invention to provide a membrane or formulation which is compostable, and more preferably biodegradable, without plastic or chemical residue(s).

It may be an object of the invention to provide a membrane, and a formulation for a membrane, that can provide a barrier to fluid(s).

It may be an object of the invention to provide a membrane, and a formulation for a membrane, that is flexible e.g. can be formed into a roll or crumpled without substantially affecting its ability to provide a barrier to fluid(s) due to cracking or damage.

It may be an object of the invention to provide a membrane that is less likely to crack or does not substantially crack when deformed and/or which is resilient.

It may be an object of the invention to provide at least one of a membrane, a formulation, and a product that includes a membrane, that is self-adhesive.

It may be an object of the invention to provide a membrane, and formulation for a membrane, that can provide a structural component for a product e.g. to bind to one or more materials to form at least a portion of the product.

It may be an objection of the invention to provide an improved sanitary product that overcomes at least some of the disadvantages with available sanitary products.

It may be an object of the invention to provide a product which includes a membrane as described herein and the benefit(s) the membrane may provide.

Alternatively, or in addition, it may be an object of the invention to provide the public with a useful choice.

### 4. SUMMARY OF THE INVENTION

According to one aspect of the invention, there is provided a membrane, wherein the membrane includes
a carrier,
a formulation provided to the carrier, and
a stabiliser.

According to another aspect of the invention, there is provided a product that comprises a membrane as substantially described herein.

According to another aspect of the invention, there is provided a new use of one or more biodegradable waxes, lipids and resins in the manufacture of a membrane, and a formulation, and a product containing a membrane.

According to another aspect of the invention, there is provided a formulation for use in manufacturing a membrane.

In embodiments, the formulation may be used to provide a fluid barrier to at least a portion of another structure .e.g a carrier. Therefore, reference will be made herein to the formulation being a “sealant formulation”. However, this should not be seen as limiting, as the formulation may not be required to seal a carrier i.e. the formulation may be used as a product in its own right as part of a product, or it may not provide a fluid barrier.

It is envisaged and the formulation may be sold as pellets, or as a relatively large volume (such as in a drum), that can be subsequently used to manufacture another product.

Throughout the present specification, reference to the term “compostable” should be understood as meaning able to breakdown under the conditions experienced in a compost environment e.g. a commercial or domestic compost system, and to comply with one or more standards for composting (as are addressed in more detail below).

Throughout the present specification, reference to the term “biodegradable” should be understood as meaning able to be breakdown under the natural action of bacteria, microbes and/or other living organisms.

It should be understood that compostability is a sub-set of biodegradability. This reflects that the standards for compostability set criteria to which a product must be broken down to be considered “compostable”, but that it may be possible to further breakdown a material despite it having met that criteria.

The present specification discusses compostability and biodegradability with reference to the sealant formulations and/or membranes according to the present technology. It should be understood that compostability and biodegradability of a membrane involves both a carrier and a sealant formulation. In preferred embodiments, the sealant formulation and carrier are both compostable and biodegradable. However, it is also envisaged that the carrier may not be compostable or biodegradable, or is so at a different rate to the sealant formulation.

In preferred embodiments, the sealant formulation and membrane may be compostable.

It should be understood that the sealant formulation and membrane may need to meet different compostability standards according to the products or applications in which they are used. However, the compostability of the sealant formulations and membrane described herein can be optimised by varying, inter alia, the composition of the sealant formulation. Therefore, the present invention and this disclosure envisages that the composition of the sealant formulation can be selected to meet a desired compostability standard or biodegradability profile.

For instance, in packaging products, the sealant formulation and membrane may meet or exceed EN1342:2000.

In particularly preferred embodiments, the sealant formulation and membrane may breakdown under normal domestic composting conditions. For instance, the normal domestic conditions may be the conditions in a home composting system.

In a particularly preferred embodiment, the normal domestic composting conditions may be as specified in one or more of EN13432:2000, ASTM D6400 or Australian standard AS5810.

In particularly preferred embodiments, the sealant formulation and membrane may breakdown in less than 60 days, and more preferably in less than 30 days.

In an especially preferred embodiment, the sealant formulation and membrane may breakdown in 18 days or less under the action of microbes and bacteria. For instance, the sealant formulation and membrane may breakdown in 18 days under normal domestic composting conditions.

Throughout the present specification, reference to the term carrier should be understood as meaning a layer of material to which a sealant formulation is applied.

In a particularly preferred embodiment, the carrier may be at least one of compostable and biodegradable.

In a preferred form, the carrier may be a structure comprising a plurality of filaments.

In a more preferred embodiment, the carrier may be at least partly formed from a fibrous material e.g. the fibrous material provides the filaments of the carrier.

In a particularly preferred embodiment, the fibrous material may be a cellulose material.

It should be understood, that in preferred embodiment, the sealant formulation can be applied to the carrier and substantially or completely prevents fluid passing through the carrier.

In a preferred embodiment, the sealant formulation may be flexible e.g. does not substantially crack when deformed, for instance when formed in a sheet and that is rolled or crumpled.

In a preferred embodiment, the sealant formulation may be applied to the carrier with a thickness of at least 25 microns.

In a particularly preferred embodiment, the sealant formulation may be applied to the carrier layer with a thickness of between 35 microns and 100 microns.

In a preferred embodiment, the sealant formulation may at least partially fill interstitial spaces between filaments forming the carrier.

In preferred embodiments, the sealant formulation may adhere to at least some of the filaments forming the carrier.

In a preferred embodiment, the sealant formulation may comprise at least one wax.

Throughout the present specification, reference will be made to wax or waxes. This should be understood as meaning mixtures of long chain lipids.

In preferred embodiments, the wax(es) may be naturally occurring waxes e.g. the wax may be selected from the list of at least one plant wax, at least animal wax, derivatives or combinations thereof.

In preferred embodiments, the wax(es) may be biodegradable as defined herein without, for example producing micro plastics or leaving undesirable residues and chemicals.

In particularly preferred embodiments, the wax(es) may be mixtures of compounds which do not contain compounds derived from petrochemicals. The mixtures may be naturally occurring mixtures, or a manmade mixture such as by mixing two naturally occurring waxes or modifying one or more naturally occurring wax.

In a particularly preferred embodiment, the sealant formulation may comprise a mixture of a first wax and a second wax.

It should be understood that reference to the first wax and the second wax are inclusive, indicating a single wax or mixture of waxes e.g. the first wax may be a mixture of two or waxes and the second wax may be a mixture of two or more waxes.

In some embodiments, the first wax and the second wax may be mixed together to form a substantially homogenous mixture.

In a preferred embodiment, the sealant formulation may comprise a mixture of a first wax having a first hardness and a second wax having a second hardness.

Throughout the present specification, reference will be made to the relative hardness of the first wax and the second wax. This should be understood as referring to the melting point of each wax compared to the other wax.

The inventor has identified that the melting point of a wax may be a proxy for other properties of the wax e.g. brittleness. For instance, a wax which has a relatively high melting point will likely be more brittle than a wax which has a relatively lower melting point. This may be useful as waxes may be mixed together to balance their physical properties. Mixing a wax with a relatively high melting point with a wax which has a relatively low melting point will produce a formulation which has melting point between that of the first wax and the second wax, and physical properties such as brittleness which is less than that of the first wax.

However, as described elsewhere in this specification, waxes with different melting points generally have low solubility with each other, and/or the mixtures have a relatively low stability over time when cooled e.g. they tend to separate. The inventor though has surprisingly discovered how to improve the miscibility of the waxes and stability of the resulting mixtures. Accordingly, the sealant formulations described herein may be better suited to different uses/applications and therefore provide advantages over the prior art.

In embodiments the first wax may be a relatively hard wax and the second wax may be a relatively soft wax.

Throughout the present specification, reference to the term hard wax should be understood as meaning a wax that has a relatively high melting point.

Throughout the present specification, reference to the term soft wax should be understood as meaning a wax that has a relatively low melting point.

The inventor has surprisingly discovered that it is possible to produce sealant formulations, and membranes made using the sealant formulations, which have desired properties such as one or more of a desired melting point, stability, resilience, and flexibility. These properties are achieved by mixtures of at least one hard wax and at least one soft wax, with a stabiliser as described herein. However, the desired properties for the sealant formulation and membrane dictate the composition of the sealant formulation. Accordingly, it should be understood that the ratio of the hard wax(es) and soft wax(es) and stabiliser (% w/w) may be adjusted to achieve those properties according to this disclosure.

In embodiments, the hard wax may have a melting point higher than the soft wax.

In particularly preferred embodiments, the hard wax may have a melting point that is substantially the same as, or above, the desired melting point of the sealant formulation.

In an even more preferred embodiment, the hard wax may have a melting point of substantially 65° C. or higher.

In embodiments, the soft wax may have a melting point that is lower than the melting point of the hard wax.

In a particularly preferred embodiment, soft wax may have a melting point of substantially 64° C. or less.

In particularly preferred embodiments, the sealant formulation may contain between substantially 25% to 40% w/w of the first wax, and more preferably substantially 30% w/w of the first wax.

The inventor has surprisingly found that these ranges for the first wax assist in providing a sealant formulation with a sufficiently high melting point that it does not melt at temperatures that may reasonably be expected in use and transport. Furthermore, the amount of the first wax can assist to provide the sealant formulation with desired structural properties such as flexibility and resilience when blended with the soft wax.

In preferred embodiments, the first wax may be selected from the list of candelilla wax, carnauba wax, palm wax, rice bran wax, sugarcane wax, sunflower wax, modifications and derivatives of the foregoing, and combinations thereof.

In particularly preferred embodiments, the sealant formulation may contain between 15-35% of the second wax, and more preferably 30% w/w of the second wax.

The inventor has surprisingly found that these ranges for the second wax assist in providing the sealant formulation with the desired flexibility, yet are stable and have a sufficiently high melting point when mixed with the hard wax. Further, the sealant formulation is not overly greasy or otherwise unpleasant to touch, which may not be the case if a higher amount of the second wax were used.

In particularly preferred embodiments, the second wax may be selected from the list of bayberry wax, coconut HO wax, hemp HO wax, Japan wax, jojoba wax, Myrica wax, rapeseed HO wax, soy wax, veewax, modifications and derivatives of the foregoing, and combinations thereof.

In a particularly preferred embodiment, the stabiliser may be a tackifier and reference will be made herein as such.

Throughout the present specification, reference to the term tackifier should be understood as meaning one or more compounds which has viscoelastic properties. For instance, the tackifier may increase the stickiness (adhesion) of a sealant formulation according to the present invention.

However, this should not be seen as limiting on the scope of the present invention as the stabiliser does not need to be a tackifier as defined herein. Instead, other forms for the stabiliser are envisaged which do not increase the stickiness (adhesion) of the sealant formulation. For instance, the stabiliser may be other compounds such as stearic acid which may improve the miscibility of the waxes, and stability of the resulting mixture.

The use of a tackifier may be particularly useful where it is desired to attach a membrane in position. For instance, one example of where a membrane may be held in position is to protect building materials such as lumber from the weather.

Another example of where a membrane could be held in position is in disposable sanitary products. For instance, a membrane according to the present invention could be included in a sanitary product, and a portion of the biodegradable membrane is positioned and/or arranged to, in use, attach the sanitary product to a person's underwear.

These aspects of the present invention should become clearer from the following description.

In a preferred embodiment, the tackifier may have a glass transition temperature above room temperature.

In preferred embodiments, the sealant formulation may comprise the tackifier e.g. at least a portion of the sealant formulation comprises a homogenous mixture of the wax(es) and the tackifier.

In particularly preferred embodiments, the substantially homogenous mixture may provide an outer surface of the membrane. This may be particularly beneficial as it can facilitate the tackifier attaching e.g. releasably attaching, the membrane to an object.

However, the tackifier could also be applied to an outer surface of the sealant formulation e.g. after application of the sealant formulation to the carrier layer. Therefore, the discussion herein should not be seen as limiting on the scope of the present invention.

In a preferred embodiment, the tackifier may have a glass transition temperature above room temperature. Using a tackifier with a relatively high glass transition temperature may assist in achieving a desired melting point for the sealant formulation. This may be particularly beneficial where the tackifier is mixed with the wax(es) to form the sealant formulation.

The inventor has surprisingly found that provision of a tackifier, particularly the tackifiers described herein, may provide a number of additional benefits. For instance, the tackifier may function as a surfactant to improve the miscibility of the first wax and the second wax with each other.

In addition, the tackifier may function as an emulsifier to assist with creating a substantially homogenous and stable mixture of the first wax and the second wax. This may be particularly helpful to assist with creating a homogenous mixture of the waxes and thereby help with creating a stable sealant formulation.

The tackifier may also function as a plasticiser, to assist with ensuring that a sealant formulation according to the present invention is relatively soft and flexible. Furthermore, the tackifier may also decrease the viscosity of the sealant formulation compared to mixtures of waxes that do not include a tackifier.

In embodiments, the tackifier may be a natural product e.g. a plant extract or plant derived substance.

In a preferred embodiment, the tackifier may be at least one of Abietic Acid, Glycerol Ester of Wood Resin, Fumaric Modified Rosin Pentaerythritol Ester, derivatives thereof or mixtures combining these compounds. The inventor has surprisingly identified that these compounds can enable the tackifier to provide the required functions e.g. visco-adhesive properties, surfactant/emulsifier, plasticiser.

In preferred embodiments, the tackifier may be a resin or a rosin, or derivative made by modification or processing of a resin or rosin. These substances are beneficial as they include one or more of Abietic Acid, Glycerol Ester of Wood Resin, Fumaric Modified Rosin Pentaerythritol Ester, and derivates, and are commercially available without requiring further processing.

In addition, these substances have relatively high melting points and so may assist in providing a sealant formulation with a desired melting point.

In a particularly preferred embodiment, the tackifier may be selected from the list of Pine rosin, Damara resin, Triethylene Glycol Rosinate (TEG), and Glycerol Ester of Wood Resin (MR), or mixtures thereof.

In a particularly preferred embodiment, the tackifier may be a food-grade compound. This may be beneficial as it can allow the biodegradable membrane to be used in food packaging, storage and preservation applications without posing health risks.

In a particularly preferred embodiment, the tackifier may be a substance that is hypoallergic, is a low-irritant or a non-irritant. These properties may make the sealant formulations and biodegradable membranes according to the present invention well suited to certain applications e.g. where they come into contact with skin or the body.

In a particularly preferred embodiment, the tackifier may be a biodegradable compound.

In a particularly preferred embodiment, the sealant formulation may comprise substantially 35% w/w to 60% w/w of the tackifier, and more preferably substantially 40% w/w of the tackifier.

It should be understood that the melting point of the sealant formulation is a key feature which makes it suitable for certain applications. Accordingly, the composition of sealant formulations according to the present invention can be adjusted to achieve a desired melting point. This should become clearer from the following description.

In preferred embodiments, the sealant formulation may have a melting point of at least substantially 45° C., and more preferably substantially 65° C., and even more preferably at least 70° C.

It is also envisaged that the sealant formulation may have a melting point of less than substantially 110° C.

These preferred melting points described herein may be useful to ensure that the sealant formulation, and products made from it, can survive the temperatures which can normally be experienced during transport and storage e.g. in shipping containers or on docks. Further, having a melting temperature of at least 45° C. may allow the sealant formulation to be used in a membrane or other product which comes into contact with a person's body, and so therefore will not melt.

According to another aspect of the invention, there is provided a sanitary product that may be degradable.

In one embodiment, the sanitary product may be biodegradable.

In one embodiment, the sanitary product may be configured to biodegrade in normal conditions in a home compost system.

In a preferred embodiment, the sanitary product may be configured to substantially biodegrade in a time period of approximately 30 to 120 days, more preferably 30 to 90 days.

In a particularly preferred embodiment, the sanitary product may be configured to fully biodegrade in a time period of less than 60 days.

In one embodiment, the sanitary product may include a body portion.

In a preferred embodiment, the body portion may be configured to adhere and bond with an absorbent material.

In a particularly preferred embodiment, the body portion may be provided by a membrane as described herein.

In one embodiment, the sanitary product may include an absorbent material.

In a preferred embodiment, the absorbent material may include fibres and gels.

In a particularly preferred embodiment, the absorbent material may be cellulose and/or derived from plants, or regenerated from plant cellular compounds.

In a further embodiment, the absorbent material may include at least one polymer, paper, tissue, felt, cloth, fabric, textile, nonwoven or woven, or a combination thereof.

In a further embodiment, the membrane may also provide a fluid-proof layer for the sanitary product.

In one form, a sealant formulation according to the present invention may permeate into the absorbent material to adhere to it and thereby attach it to the carrier layer.

In a further aspect of the invention, the membrane may be adapted to provide structural bonding of components in the sanitary product; for example to adhere and bond two or more components of the sanitary product.

In a further embodiment, the membrane may be adapted to fluid-proof the sanitary product to, for example, retain fluid within the sanitary product and to thereby reduce or eliminate leaking.

In another embodiment, the adhesive layer may be adapted to provide adhesion between layers of absorbent materials within the sanitary product. For example the adhesive layer may adhere layers of absorbent materials together in the sanitary product.

In a particularly preferred embodiment, the membrane may be adapted to provide two or more of the functions of structural bonding of components, fluid-proofing and adhesion between layers of absorbent material.

The membranes of the present invention, and products comprising the membranes, may provide a number of advantages over the available membranes.

For instance, the sealant formulation may be comprised of a combination of natural waxes, lipids and/or resins. These compounds are selected to provide a fluid barrier which, when exposed to environmental conditions or a composting environment, will break down within a designated time frame. As a result, the biodegradable membranes described herein may minimise or resolve the issues of waste and environmental damage caused by single use plastic products such as plastic membranes and sheets.

Furthermore, in embodiments, the carrier is a biodegradable material such as cellulose and it is configured to biodegrade relatively rapidly. Therefore, appropriate identification of the carrier also assists to minimise issues of waste and environmental damage caused by single use plastic products such as plastic membranes and sheets.

Together, the carrier and sealant formulation of the present invention have surprising performance. The inventors have surprisingly found that the membranes of the present invention can be deformed such as by rolling or crumpling, without substantially or completely affecting their ability to provide a fluid barrier.

Furthermore, the sealant formulation can provide an effective fluid barrier at a range of different thicknesses. Without being limited to a specific mechanism, the inventors tentatively suggest that the sealant formulation may fill the interstices between filaments forming the carrier and adhere to the filaments. As a result the sealant formulation may provide more than only a superficial barrier.

The inventors have surprisingly found that the provision of a sealant formulation comprising a mixture of soft and hard waxes as described herein can provide an effective fluid barrier. In addition, the mixtures of waxes may provide better surface finishes such as being less prone to cracking than other ratios or combinations of waxes. Furthermore, the mixtures of waxes described herein may provide biodegradable membranes which are stable are a desired range of temperatures, which in turn makes them suitable for certain uses.

The inventors have also surprisingly found that mixing a tackifier into the sealant formulation does not adversely affect the stability or other properties of the sealant formulation. This allows a single formulation to be applied to a carrier to provide both fluid proofing and adhesion functions for the membrane in use.

While uncertain, the inventors also suggest that inclusion of the tackifier in the sealant formulation may assist with mixing and stability of the sealant formulation, and/or allowing an optimum ratio of waxes to provide the required functions of the sealant formulation.

Further aspects of the invention, which should be considered in all its novel aspects, will become apparent to those skilled in the art upon reading of the following description which provides at least one example of a practical application of the invention.

### 5. BRIEF DESCRIPTION OF THE DRAWINGS

One or more embodiments of the invention will be described below by way of example only, and without intending to be limiting, with reference to the following drawings, in which:

FIG. 1 is a cross sectional view of a biodegradable membrane in accordance with one embodiment of the present invention.

FIG. 2 is a cross sectional view of a biodegradable membrane in accordance with another embodiment of the present invention.

FIG. 3 is an end on view of a biodegradable membrane in accordance with one embodiment of the present invention arranged in a roll.

FIG. 4 is a top view of a sanitary product in accordance with one embodiment of the present invention.

FIG. 5 is a cross section view of a sanitary product in accordance with an embodiment of the present invention.

FIG. 6 is a cross sectional view of a sanitary product in accordance with an embodiment of the present invention.

FIG. 7 is a bottom view of a sanitary product in accordance with an embodiment of the present invention.

FIG. 8 is an exploded view of a sanitary product in accordance with an embodiment of the present invention.

FIG. 9 is a perspective view of a sanitary product in accordance with an embodiment of the present invention.

FIG. 10 is a perspective view of a sanitary product in accordance with an embodiment of the present invention.

FIG. 11 is an exploded view of a sanitary product in the form of a diaper in accordance with an embodiment of the present invention.

FIG. 12 is a cross sectional view of a sanitary product in the form of a wound dressing in accordance with an embodiment of the present invention.

FIG. 13 is a cross sectional view of a sanitary product in the form of a wound dressing in accordance with an embodiment of the present invention.

FIG. 14 is a cross sectional view of a sanitary product in the form of a medical underlay in accordance with an embodiment of the present invention.

FIG. 15 is a cross sectional view of a sanitary product in the form of an animal bed in accordance with an embodiment of the present invention.

FIG. 16 is a photograph of the sanitary products used in the compost trial at day 0.

FIG. 17 is a photograph of the sanitary products used in the compost trial at day 0.

FIG. 18 is a photograph of the top side of sanitary products used in the compost trial at day 32.

FIG. 19 is a close-up photograph of the top side of sanitary products used in the compost trial at day 32.

FIG. 20 is a photograph of the underside of sanitary products used in the compost trial at day 32.

FIG. 21 is a photograph of the corn starch sheet used in the compost trial at day 32.

FIG. 22 is a flow diagram showing representative steps in a method of manufacturing a sanitary product in accordance with an aspect of the invention.

FIG. 23 is a table showing representative sealant formulations according to an aspect of the invention and tests of their physical properties.

FIG. 24 is a table showing representative sealant formulations according to an aspect of the invention and tests of their physical properties.

FIG. 25 is a table showing representative sealant formulations according to an aspect of the invention and tests of their physical properties.

FIG. 26 is a table showing representative sealant formulations according to an aspect of the invention and tests of their physical properties.

FIG. 27 is a table showing representative sealant formulations according to an aspect of the invention and tests of their physical properties.

FIG. 28 is a table showing representative sealant formulations according to an aspect of the invention and tests of their physical properties.

FIG. 29 is a table showing representative sealant formulations according to an aspect of the invention and tests of their physical properties.

### 6. DETAILED DESCRIPTION OF PREFERRED EMBODIMENTS OF THE INVENTION

6.1 Formulation Design

The inventor has surprisingly identified how to manufacture membranes which may be one or more of flexible, waterproof, structurally coherent, stable, has a desired melting point, and has inherent adhesive properties. The present invention and disclosure herein can be used to define the required functional properties for the membrane, and then to create a formulation which can be used to manufacture the membrane and achieve the required properties.

In preferred forms, the invention is enabled by a unique blend of two or more waxes and at least one other compound e.g. a stabiliser as described herein. The two or more waxes may be a relatively hard wax and a relatively soft wax.

Surprisingly, the inventor has discovered that the use of a stabiliser, and more preferably a tackifier, as described herein can assist in achieving a formulation with physical properties that may be required for certain applications. For instance, the stabiliser can provide an adhesive function (e.g. it acts as a tackifier) to assist with adhering a membrane to another object (or itself). It may also assist with adhering the formulation to a carrier as described herein which can assist in manufacturing a membrane according to the forms of the invention.

The stabiliser may also assist in achieving a desired melting point for the formulation. For instance, the stabiliser may have a relatively high melting point e.g. a melting point that is higher than the melting point of at least the relatively soft wax, and preferably also the relatively hard wax.

In addition, the tackifier may act as a surfactant e.g. such as an emulsifier. For instance, the tackifier may promote the relatively hard wax and the relatively soft wax being mixed after melting to create a substantially homogenous mixture. The tackifier may then act to stabilise the mixture of waxes helping to prevent them separating on cooling of the homogenous mixture.

In some embodiments, the tackifier may also function as a plasticizer, helping to provide a desired degree of flexibility and/or softness to a sealant formulation according to the present invention.

In some embodiments, the invention can be considered to comprise a method which includes one or more of the following steps in any order:
1. Defining desired physical properties for a formulation. The physical properties may be selected from the list of one or more of melting point, flexibility, colour, adhesion,
2. Selecting a first wax which has a first hardness;
3. Selecting a second wax which has a second hardness;
4. Melting the first wax to create a first molten wax;
5. Melting the second wax to create a second molten wax;
6. Causing the first molten wax and the second molten wax to mix to create a substantially homogenous mixture;
7. Selecting a stabiliser;
8. Melting the stabiliser;
9. Mixing the stabiliser with the first wax and/or the second wax or the homogenous mixture, to create a formulation;
10. Cooling the formulation.

As described elsewhere in this specification, the first wax and the second wax preferably have different melting points to each other e.g. the first wax has a relatively high melting point and the second wax has a relatively low melting point.

In addition, the stabiliser preferably has a relatively high melting point e.g. preferably higher than the melting point of the second wax, and more preferably higher than the first wax. As a result, the stabiliser may assist to increase the melting point of the formulation above the individual melting points of the first wax and the second wax.

A desired melting point for the formulation can therefore be achieved by selecting the relative proportions of first wax, the second wax, and the stabiliser, or the specific waxes or tackifier(s) used. In addition, other desired properties of the formulation can be achieved by varying the waxes and tackifiers used, and the relative proportions of each.

6.2 Membranes

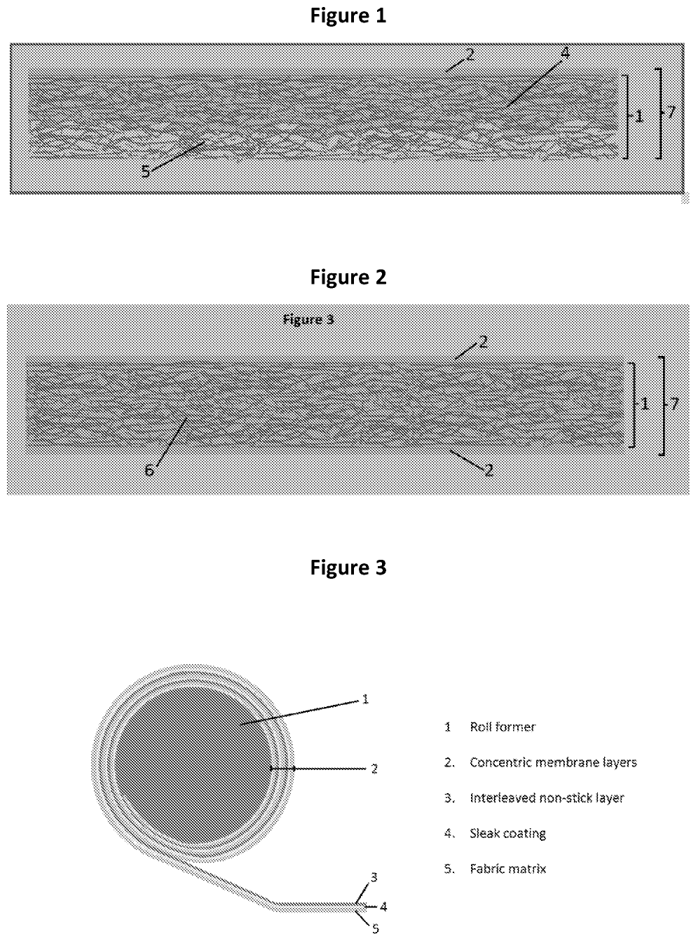

Referring to FIGS. 1-3 which show views of a membrane 100 according to one aspect of the present invention.

The membrane includes a carrier indicated generally as 102, and a sealant formulation indicated generally as 104.

The formulation 104 is applied to the carrier 102 using any suitable technique such as those discussed in more detail below.

In the embodiment of FIG. 1, the formulation provides surface coating for the carrier 102, and only partially penetrates into the carrier 102. As a result, a surface of the carrier 102 may be exposed.

In the embodiment of FIG. 2, the formulation 104 penetrates through the entire thickness of the carrier 102. As a result, the carrier 102 is substantially or completely covered by the formulation 102.

The membrane 100 according to the present invention can be deformed. However, deformation does not substantially or partially affect the ability of the formulation to provide a fluid barrier.

Examples of the type of deformation which the membrane 100 can withstand including rolling or crumpling. These deformations do not affect the ability of the formulation 102 to provide a fluid barrier. For instance, the formulation may not crack on deformation which means that there are no fissures through which a fluid can seep from one side of the membrane 100 to the other. Alternatively, if the sealant formulation 104 does crack then penetration of the formulation 104 into the interstices between fibres forming the carrier 102 may provide a sufficient fluid barrier.

Further aspects of the carrier 102 and the formulation 104 should become clearer from the following description.

6.2 Sanitary Product Including a Membrane

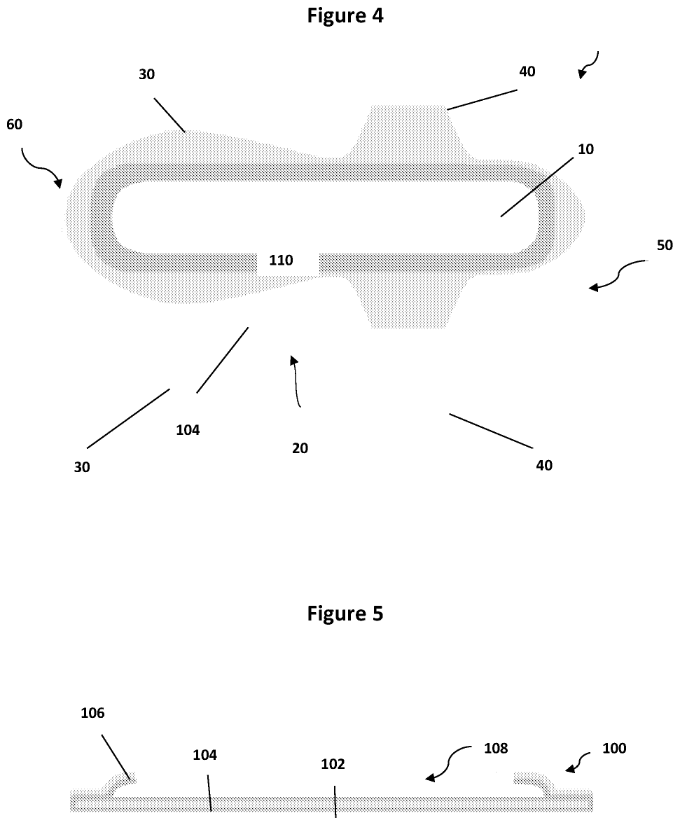

Referring now to FIGS. 4-6 which show use of a membrane according to an aspect of the present invention used to manufacture a product in the form of a sanitary product (100) in the form of a menstrual pad. Reference to a menstrual pad is by way of example only, and the present invention should not be seen as being limited to such. The sanitary product (100) is adapted to biodegrade e.g. in the normal conditions within a domestic composting system. For instance, the sanitary product (100) may biodegrade in the conditions specified in EN 13432, ASTM D6400.

However, it will be appreciated that the sanitary product (100) will also be fully biodegradable under other conditions e.g. in industrial composting environments, or in the natural environment.

The sanitary product (100) includes a body portion (20) which is formed includes at least a formulation (104). The sealant formulation (104) is discussed in further detail below.

In the illustrated embodiments, the body portion (20) is a multilayer structure which comprises the formulation (104) in combination with at least one other layer (as discussed in more detail below).

As illustrated in FIG. 4, the body portion (20) comprises a central portion (10), a pair of wings (40) located at or towards a first end (50), and a skirt (30) which extends from the lateral sides of the central portion (10) at or towards a second end (60).

The sanitary product (100) has a multi-layer construction and is formed from multiple layers of materials. The multi-layer construction is perhaps best illustrated in FIGS. 2-3, which shows a cross-sectional view of the sanitary product (100). FIG. 5 shows an exploded view of the sanitary product (100) showing the layers forming the multi-layer construction.

The sanitary product includes a core (102). The core (102) may be any suitable material. However, in the preferred embodiments, the core (102) is made from a biodegradable material e.g. an organic cotton sheet.

The core (102) is preferably, soft, flexible and porous to facilitate bonding to at least one other layer of the sanitary product (100).

It should also be appreciated that the sanitary product (100) may not include a core (102).

A layer of the formulation (104) may be applied to the core (102) As can be seen in FIG. 7, the formulation (104) is provided to coat and define the entire underside of the sanitary product (100).

In addition, the formulation (104) wraps around the lateral and end edges of the core (102) and also coats the entire upper side of the core (102).

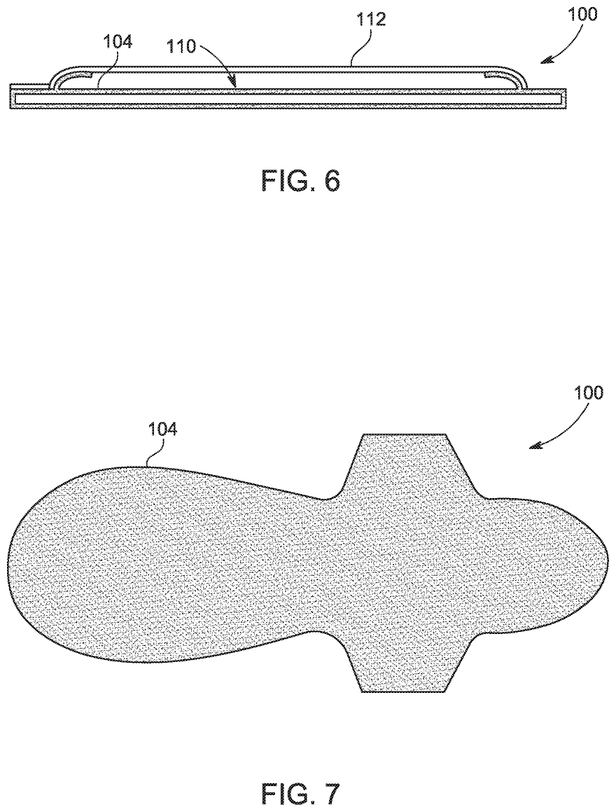

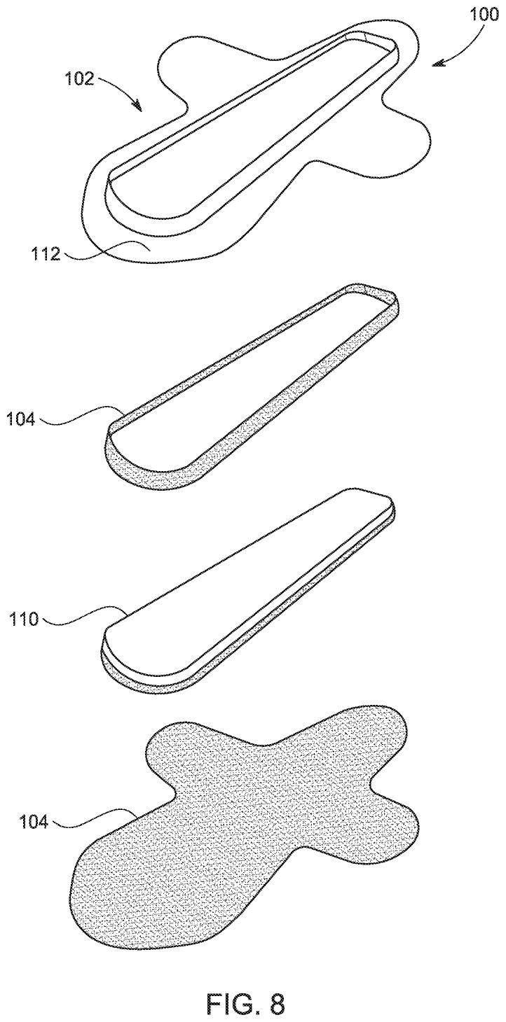

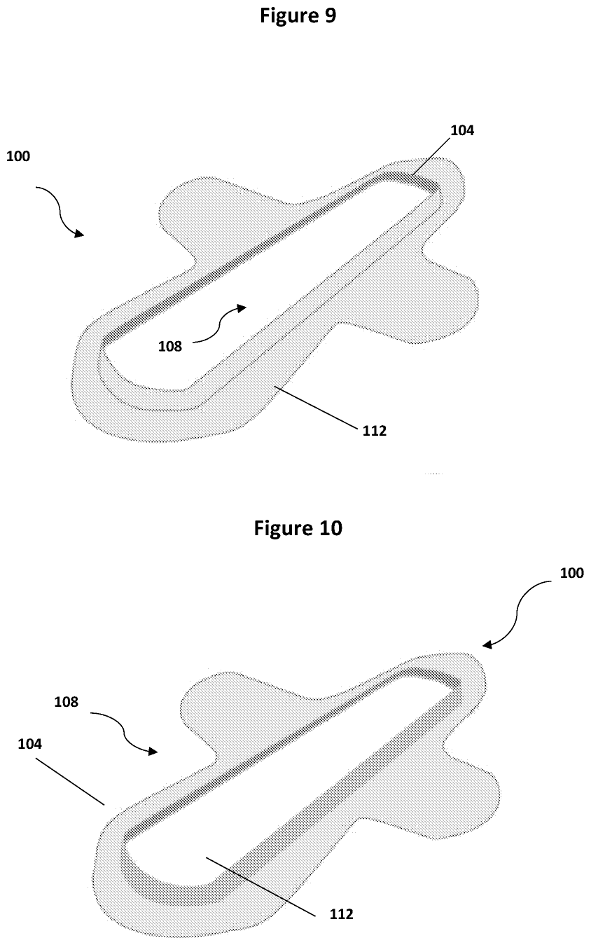

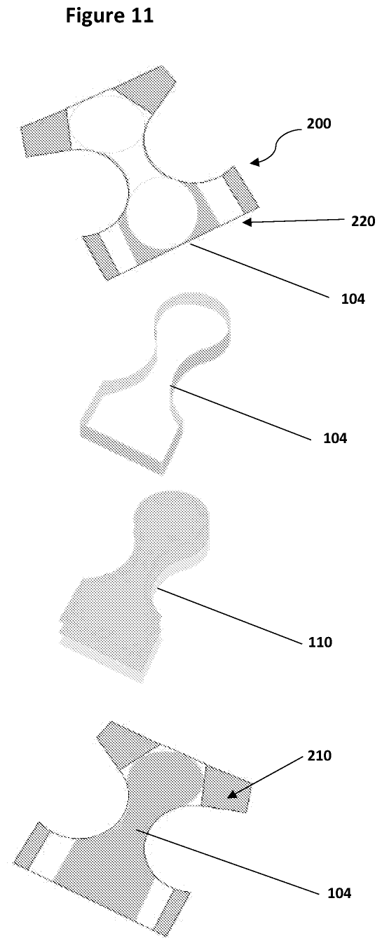

The formulation (104) is also shaped to defined flanges (106) on the upper side of the core (102). The flanges (106) extend in a shape e.g. an oval, rectangular or other shape, and define a cavity which is indicated generally as (108) in FIGS. 5-6.

The cavity (108) is shaped to received and hold an absorbent material, indicated generally as (110) in FIG. 4.

Aspects of the formulation (104) and its composition are discussed in more detail below.

The sanitary product (100) also includes an upper coating layer (112). The upper coating layer (112) may be any material e.g. an organic cotton fabric, or an absorbent, distributive, retentive. However, in preferred embodiments, the upper coating layer (112) may be made from a material which is at least one of biodegradable, soft, flexible, biocompatible, breathable, porous.

It should be appreciated that the sanitary product (100) has been described in relation to a menstrual pad but it should not be seen as limited to such. The present invention can be readily applied to other sanitary products such as incontinence wear, infant diapers, medical underlays, veterinary underlays, pet urinary training pads, and other sanitary items which absorb and retain fluid contents.

The body portion (20) is adapted to provide one or more of the following functions:
(a) structural bonding of one or more components forming the sanitary product (100);
(b) fluid-proofing and leak prevention;
(c) adhesion of absorbent material together into a mass;
(d) adhesion of the sanitary product (100) in a position in use e.g. to a person or in a wearer's underwear.

In particularly preferred embodiments, the body portion (20) is configured to provide all of the above functions. For instance, the shape and construction of the body portion (20) and the materials from which it is constructed e.g. the sealant formulation, enable it to perform all of the above functions.

The body portion (20) may be provided to cover a section of the menstrual pad or an outer layer of the absorbent material as shown in FIGS. 5-7. In either case it will be appreciated that the body portion (20) will extend substantially continuous across the whole or a substantial part of, the external outer layer of the pad, including the “wings” and “skirts” as shown in FIGS. 1-4.

The body portion (20) may also be configured to form a waterproof film on the sanitary pad and thereby provide a fluid-proof layer on an exterior surface of the sanitary product (100). In use, the body portion (20) may be used in place of stitching, felting or gluing to adhere and bond the internal components of the sanitary product (100) such as the absorbent to each other.

In the case of menstrual pads, the outer cover may comprise a bottom layer that is fully-sealed with the sealant formulation on both surfaces and an upper-layer with only the bottom surface thereon sealed in order to provide a water-proof layer. The upper or inner surfaces next to a wearer's skin need not be sealed but may remain uncoated as soft fabric or the like to provide added comfort to the wearer.

The body portion (20) may comprise any mixture of lipid or resinous compounds that is stable in solid form at temperatures from approximately 0° C. to approximately 50° C. Preferably, across the range of 35° C. to 45° C. it must be cohesive but remain flexible.

The sealant composition may also provide adhesive qualities sufficient for releasably fixing the pad in a person's undergarments and/or onto surfaces and substrates such as operating tables. The use of, for instance, resin, gum or latex or the like may provide the noted adhesive qualities.

It will be appreciated that the term absorbent material may include any suitable components or materials capable of absorbing liquids and/or bodily fluids. For example, the absorbent material may include absorbent or superabsorbent fibres and gels, based on cellulose and derived from plants directly, or regenerated from plant cellular compounds, and/or may include types commonly referred to as polymer, paper, tissue, felt, cloth, fabric, textile, nonwoven or woven.

The absorbent material is biodegradable.

It should be appreciated that the sealant formulation (104) is intended to use only physiologically acceptable compounds and can include any lipid, wax, resin and related compounds or derivatives therefore. For example, a suitable resin derivative may include a triethylene glycol, such as a triethylene glycol ester of resin.

The sanitary product (100) of the present invention is designed to be fully biodegradable under normal domestic composting conditions. However, it is also envisioned that the sanitary product (100) will also be fully biodegradable under industrial composting processes. Under either method, the present invention is configured to fully biodegrade rapidly so as to reduce harmful emissions and/or toxic residues upon degradation. It will be appreciated that a domestic composting environment may be more readily available to a consumer/user rather than industrial composting and as such it may provide a more practicable solution for the consumer to use such systems to compost the sanitary product(s) of the present invention.

Additionally, domestic composting generally requires little effort to maintain. Therefore, the sanitary product (100) according to the present technology may also encourage more people to start their own domestic composting system, however it should be appreciated that this may be seen as an optional further added advantage arising from the present invention.

Generally, industrial composting systems are far larger in scale and requires more efficient control of environmental conditions such as temperature, moisture, and airflow to ensure optimal composting.

A number of domestic composting systems are known and widely available. Generally, under such systems, a composting bin is used. The bins come in a variety of shapes and sizes and all can be used with the present invention.

The bin is generally located in a sunny position in order to provide heat to the composting system. Composting material in the form of garden and/or food waste can be added to the composting bin. The sanitary product of the present invention can be used in such systems.

In general, it will be appreciated that a sanitary product of the prior art include three main components, being at least one layer of an absorbent material(s) formulated to receive and retain fluids, adhesives to adhere layers of the absorbent material in the sanitary product and/or adhere to a user, an outer covering or coating designed to enclose the absorbent material and prevent escape of the retained fluids.

Typically, the outer coverings are composed of a plastic film. In a menstrual pad, the outer covering/coating extends around the absorbent area of the pad in the form of “wings” or skirts” to improve the wearer's comfort.

The plastic films commonly used are generally oil-based synthetics that do not biodegrade or leave toxic residues upon degradation. By its inherent function, the outer covering/coating is required to be waterproof and designed to withstand leakage from the absorbent material. However, the use of plastics materials leads to creation of significant amounts of non-biodegradable waste or in some cases micro-plastics.

In contrast the sanitary product of the present technology may be significantly, or totally, free of non-biodegradable materials or micro-plastics. As a result, the present technology significantly reduces or eliminates waste created through use of sanitary products and other disposable sanitary products.

6.3 Alternative Products

It will be appreciated by the person skilled in the art that the present invention can be applied to a wide range of products, and therefore is not limited only to those products such as menstrual pads that are described herein. Other representative products in which the present invention could be incorporated include absorbent pads, absorbent sanitary articles other than menstrual pads, absorbent sanitary clothing, absorbent protective liners for underclothing, absorbent incontinence pads, absorbent incontinence diapers, absorbent bandages, absorbent nursing (breast feeding) pads, absorbent nappies for babies [diapers], absorbent liners for diapers, sanitizing wipes, absorbent pads for medical purposes, absorbent sheets for veterinary purposes, absorbent sheets and underlays for beds, absorbent sheets and underlays for animal care, absorbent underlays for animal transportation and the like. Selected embodiments of representative products are shown in FIGS. 11-15.

Aspects of the products illustrated in FIGS. 11-15 are similar to those of the sanitary product (100), and therefore like references refer to like components/features.

FIG. 11 illustrates a diaper (200) according to the present invention. The diaper (200) includes an exterior layer (210) and at least one absorbent layer (110). The formulation (104) may be applied around the edges of the absorbent layer(s) (110) and exterior layer of the diaper to bond the internal absorbent layer(s) together and fluid proof the diaper (200) respectively. The formulation (104) may also be applied on the exterior layer of the diaper to provide self-fastening properties.

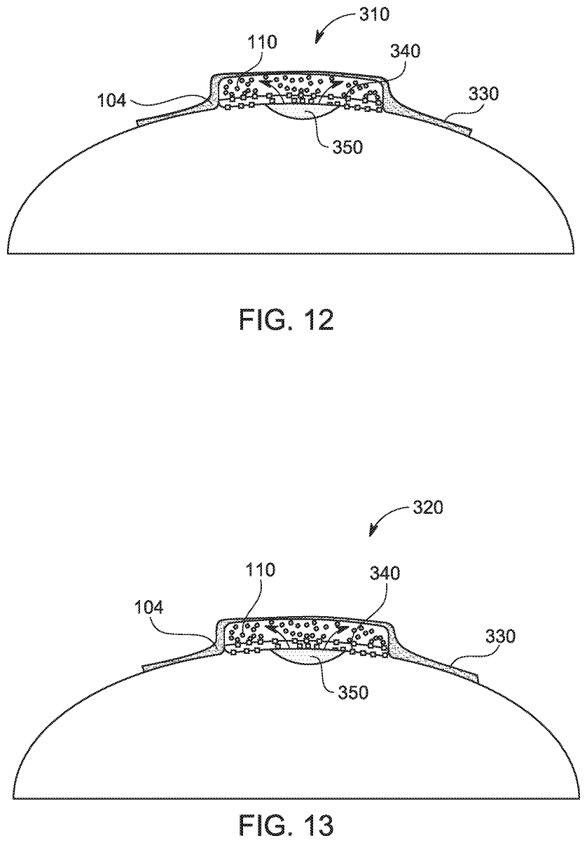

With reference to FIGS. 12 and 13, an absorbent wound dressing (300) is shown. The wound dressing may be configured as a breathable dressing (310) or as a sealed dressing (320). The wound dressings (310 and 320) each include an outer fabric cover (330), at least one absorbent layer (110) and an internal lining fabric (340). The arrows generally represent the direction of fluids drawn from the wound site (350).

The formulation (104) is provided to the wound dressing and may either substantially cover the absorbent layer(s) and internal lining (340) to provide a sealed dressing (320) or alternatively, may be included on either side thereof to provide for a breathable dressing (310). In such a configuration, the formulation has been applied to provide bonding between the outer fabric cover (330) with the absorbent layer(s) (110) and internal lining fabric (340); and further provides fluid and/or air proofing properties to the wound dressing.

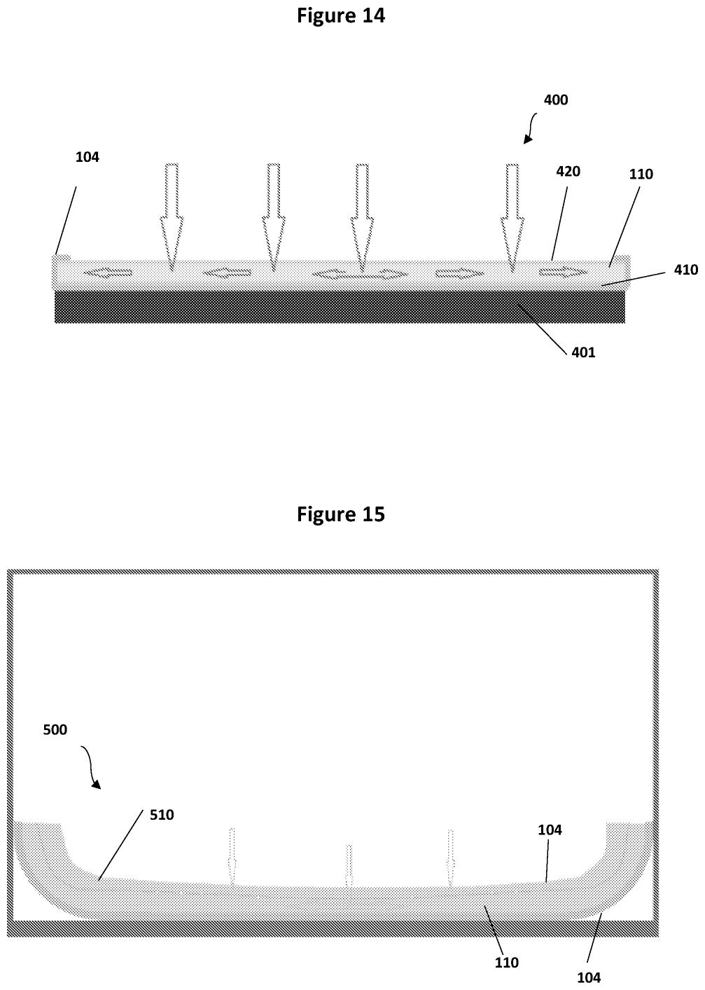

FIG. 14 shows a medical/surgical grade underlay (400) as placed on a bench top (401). The underlay includes a bottom cover-layer (410), an absorbent layer(s) (110) and a top cover (420). The arrows generally represent the fluids entering into the underlay (400). The formulation (104) is applied to enclose the bottom cover-layer, absorbent layer and top cover to provide a fluid proof the underlay (400) to ensure fluid is retained therein and also bond the layers together within the underlay (400).

With reference to FIG. 15, an animal bed (500) is shown. The animal bed includes a top fabric layer (510) and an absorbent layer (110). The top fabric layer is configured to allow fluid matter to pass through it and into the absorbent layer (110)—as generally represented by the arrows. The formulation (104) is applied under the absorbent layer to provide a fluid-proof seal to ensure fluid retention, bonding to the absorbent layer and safe hygienic handling. The formulation (104) may also be applied in a non-continuous form between the top fabric layer and absorbent layer to bond the two respective layers together. The non-continuous application of the formulation in this configuration allows for any fluid to pass through from the top fabric layer (510) into the absorbent layer (110).

It should also be appreciated that the present invention may also find further application in other products which do not include an absorbent material. It will be appreciated that the invention provides fluid-proofing, adhesion and bonding of materials, as such may be applied to suitable products where such properties are desired. For instance, suitable products include sanitary articles; non-absorbent bandages; non-absorbent sheets and underlays; non-absorbent articles of attire; non-absorbent bags and containers; non-absorbent food packs; non-absorbent food-waste containers and the like. In these embodiments, the sealant formulation (104) will provide the functions of fluid-proofing, adhesion in place during use, and to bind the layers of the product together.

6.4 Representative Method of Manufacture

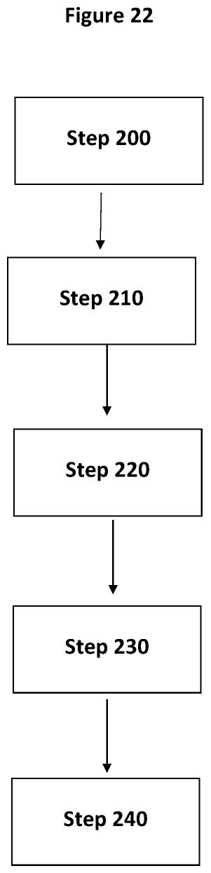

Referring now to FIG. 22 which shows representative steps in a method of manufacturing the sanitary product (100) according to an aspect of the invention.

At step 200 the core (102) is formed. Suitable methods to form the core include cutting a predefined shape from a sheet of material using either a continuous or discontinuous method.

At step 210, the sealant formulation (104) is applied to the core (102). This may occur using any suitable technique such as spraying dipping, rolling, die spreader

Step 220 preferably completely coats the core with the sealant formulation (104). In doing so, the sealant formulation (104) wraps around the lateral sides and ends of the core, to completely coat the underside, upper side, edges and ends of the core (102). In addition, step 220 also forms flanges (106) to define the cavity (108).

It is also envisaged that the sealant formulation may be applied to the core (102) as a powder or granules, and subsequently heated.

At step 230 the upper coating layer (112) is applied to the upper surface of the formulation (104).

At step 240, the absorbent is provided to the cavity.

Optionally, a heating step may be used to at least partially melt the formulation (104) to promote adhesion to one or more of the core (102), the upper coating layer (112), and the absorbent.

In each of the above noted application methods, it will be appreciated by the person skilled in the art that any one of, or a combination thereof, may be used in accordance with the best industrial practice in the manufacture of the preferred sanitary product.

6.5 Example Formulations for the Sealant Formulation

The present invention will now be described by reference to the following example formulations for the sealant formulation (104). However, such examples should not be seen as limiting on the scope of the present invention.

(a) First Representative Formulation

| | Amount | |
|---|---|---|
| | (w/w) | Comments |
| Palm Wax | 60% | |
| Coconut Wax (HO) | 15% | |
| Pine Rosin | 25% | Combine both Palm and Coconut wax |
| | | together and apply to required fabric |
| | | layer at substantially 120° C. |

(b) Second Representative Formulation

| | Amount | |
|---|---|---|
| | (w/w) | Comments |
| Palm Wax | 30% | |
| Coconut Wax (HO) | 30% | |
| Pine Rosin | 40% | Combine both Palm and Soy wax |
| | | together and apply to required fabric |
| | | layer at substantially 120° C. |

(c) Third Representative Formulation

| | Amount | |
|---|---|---|
| | (w/w) | Comments |
| Beeswax | 70% | |
| Pine Rosin | 30% | Combine both Beeswax and Pine rosin |
| | | together and apply to required fabric |
| | | layer at substantially 120° C. |

(d) Fourth Representative Formulation

| | Amount | |
|---|---|---|
| | (w/w) | Comments |
| Microcrystalline | 66% | |
| wax | | |
| Pine Rosin | 34% | Combine both microcrystalline wax |
| | | and Pine rosin together and apply to |
| | | required fabric layer at |
| | | substantially 120° C. |

(e) Fifth Representative Formulation

| | Amount | |
|---|---|---|
| | (w/w) | Comments |
| Palm wax | 30% | |
| Soy wax | 30% | |
| Triethylene | 40% | Combine both palm wax, soy wax and |
| glycol ester | | triethylene glycol ester together and apply to |
| of resin | | required fabric layer at substantially 120° C. |

(f) Sixth Representative Formulation

| | Amount | |
|---|---|---|
| | (w/w) | Comments |
| Palm Wax | 30% | |
| Soy Wax | 30% | |
| Pine Rosin | 40% | Combine both Palm and Soy Wax |
| | | together with the Pine Rosin and apply to |
| | | required fabric layer at substantially 120° C. |

(g) Seventh Representative Formulation

| | Amount | |
|---|---|---|
| | (w/w) | Comments |
| Sugarcane Wax | 25% | |
| Soy Wax (HO) | 25% | |
| Pine Rosin | 50% | Combine both Sugarcane and Soy wax |
| | | together with the Pine Rosin and apply to |
| | | required fabric layer at substantially 120° C. |

(h) Eight Representative Formulation

| | Amount | |
|---|---|---|
| | (w/w) | Comments |
| Beeswax | 70% | |
| Pine Rosin | 30% | Combine both Beeswax and Pine Rosin |
| | | together and apply to required |
| | | fabric layer at substantially 120° C. |

(i) Ninth Representative Formulation

| | Amount | |
|---|---|---|
| | (w/w) | Comments |
| Biodegradable | 66% | Combine both microcrystalline wax and |
| Microcrystalline | | Pine Rosin together and apply to required |
| Wax | | fabric layer at substantially 120° C. |
| Pine Rosin | 34% | |

(j) Tenth Representative Formulation

| | Amount | |
|---|---|---|
| | (w/w) | Comments |
| Rice-bran Wax | 30% | |
| Soy wax | 30% | |
| Abietic Acid | 40% | Combine Rice-Bran wax, Soy wax and |
| | | Abietic Acid together and apply to required |
| | | fabric layer at substantially 120° C. |

In the above formulations, it should be noted that the components may be applied to the required carrier by use of a roller, spray, brush, immersion or any other means.

It should be appreciated by the person skilled in the art that further biodegradable formulations using one or more materials selected from the list of waxes, oils, latexes, resins, and gums, and/or other derivatives/materials can be used with the present invention. The principles of fluid-proofing, bonding and adhesion in the present invention can be applied across all such formulations in application to disposable sanitary products.

6.7 Exemplification

6.7.1 Biodegradation Trial

A sanitary product (100) in accordance with the present invention was used in this trial. The sanitary product (100) was constructed using conventional absorbent materials obtained from an existing product. A purpose-made outer casing was used to house the absorbent material.

For the purposes of the present trial, the nature and performance of absorbent material was not reviewed as it was only representative of conventional absorbent material used in the industry.

The formulation (104) was made from a formulation comprising a mixture of plant-originated compounds intended to perform three concurrent functions in disposable hygiene pads: (a) structural bonding; (b) fluid-proofing of the exterior; (c) adhesion between the pad and a substrate (in menstrual use, an undergarment).

The purpose of the trial was to compare the rate and type of breakdown of a sanitary product of the present invention against that of a commercially available product marketed to be environmentally-friendly, under the brand name “Bamboo Babe”. The comparison is claimed to be made from 100% bamboo.

6.7.2 Fluid Absorption Capacity Testing

Menstrual pads made in accordance with the present invention were tested for the capacity to absorb moisture. They were filled with a dye-water solution until saturated, their weight changes were recorded and any leakage noted. They were then left for 18 hours and checked again for leakage. No leakage was evident. The results of which follow:

| | Before weight | Saturated weight | |
|---|---|---|---|
| Sample # | (g) | (g) | Leakage |
| 1 | 14 | +120 mL = 134 gm | No |
| 2 | 14 | +120 mL = 134 gm | No |
| 3 | 14 | +120 mL = 134 gm | No |
| 4 | 14 | +120 mL = 134 gm | No |
| 5 | 14 | +120 mL = 134 gm | No |

From this test, pad 3 was used for the compost testing together with the competing product noted above and a sheet of corn starch packaging. All test pads were placed on fresh grass-clippings in a standard domestic compost bin on the same day and covered with part-decayed compost, then layered weekly with fresh vegetable scraps, grass clippings and leaves under newspaper sheets for approximately 32 days.

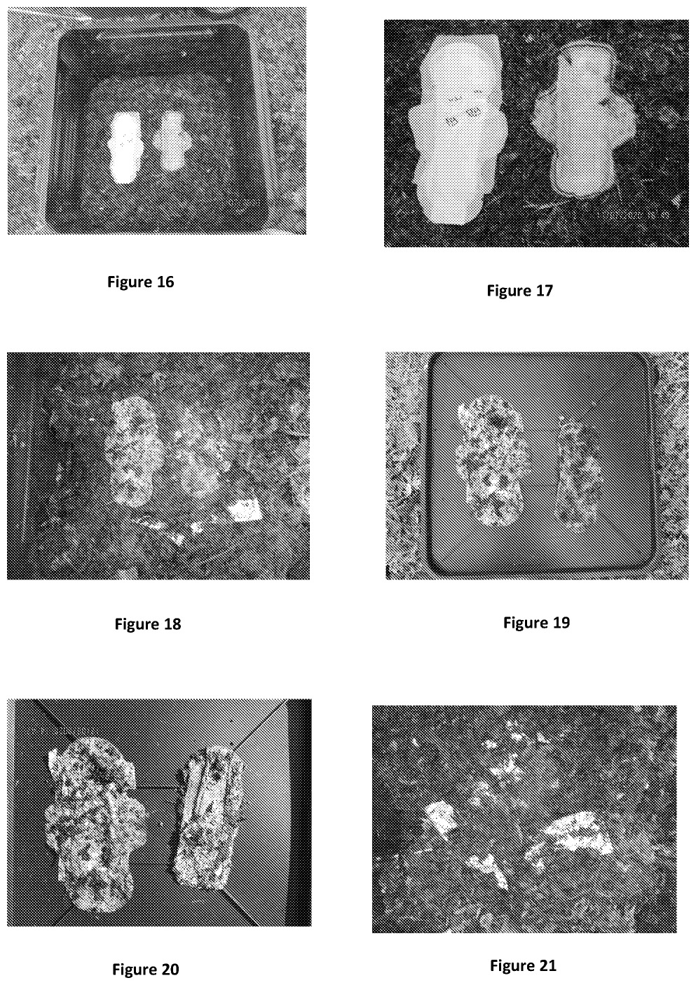

Photographs were taken at the start (FIGS. 16 and 17) and end (FIGS. 18 to 21) of the trial.

6.7.3 Ambient Conditions

It should be noted that the trial was conducted in the middle of winter. Composting ideally takes place in warm conditions and it is generally recommended that bins are placed in sunny locations to speed up bioactivity. This bin used in the trial was located in a shaded corner and during the month-long trial temperatures recorded were at times below 0° C., rising during daytime to about 15° C. maximum. Therefore, the tests conditions for this trial were not considered to be optimal conditions for biodegradation.

6.7.4 Observations

After day 32 of the trial, it was observed that:
the competing product emerged largely unchanged. Its paper wrapping had disappeared but the pad itself was structurally intact, its coating was still mechanically functional and surface details were distinct.
The corn starch sheet was also largely unchanged, although broken during uncovering and deeply stained.
The sanitary product (100) of the present invention (100) had lost its wings, skirts and the entire adhesive formulation layer on both surfaces except for small shreds. The absorbent material remained intact.

By contrast with the competing product's fluid-proofed parts, which remained largely intact and undegraded, the present invention's proofing had completely biodegraded in one month under substandard winter composting conditions.

This suggests the present invention provides functional fluid-proofing for a duration two to three times as long as probable maximum use (˜18 hours use), but biodegrades very rapidly in natural conditions and, having no toxins or artificial compounds in its ingredients, is unlikely to have left any undesirable residues.

6.7.3 Formulation Assessments

The inventor has trialled different formulations for the sealant formulation according to the present technology. These trials included preparing multiple formulations which include different with proportions for the first wax, second wax, and stabiliser (% w/w) to each other, and using those to manufacture a membrane. Selected physical properties of the membranes were subsequently tested, to assess the efficacy of the present technology.

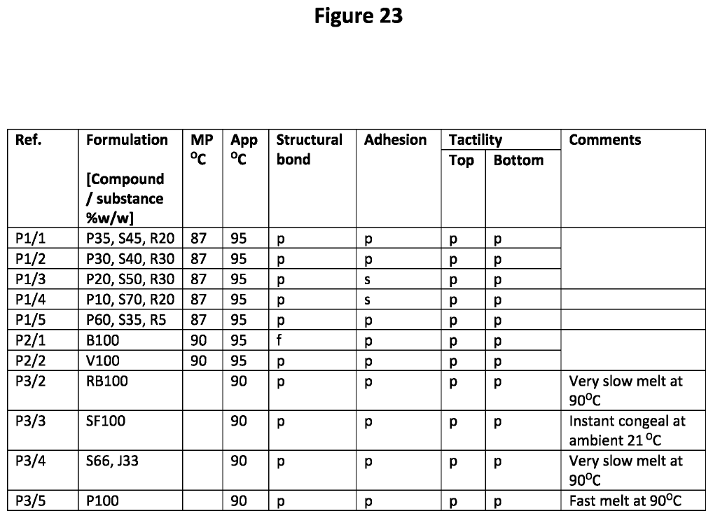

A selection of the formulations trialled, and the results of physical tests, are outlined in FIG. 23. FIG. 23 also includes results of tests of physical properties of single waxes or mixtures of a single wax and stabiliser in the form of a tackifier, by way of comparison to the present technology.

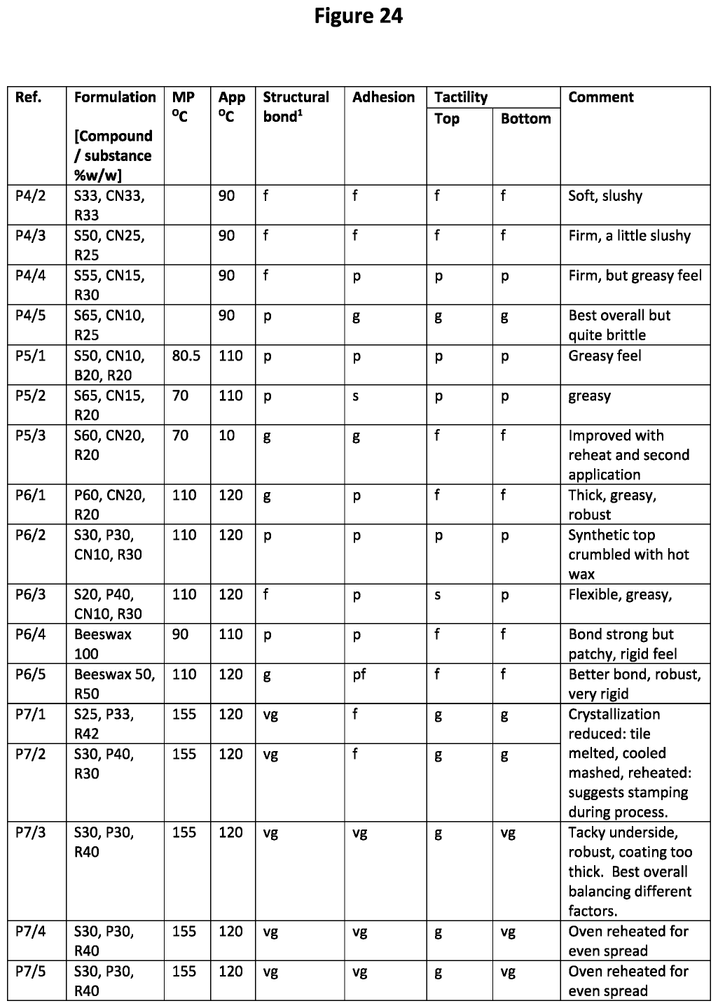

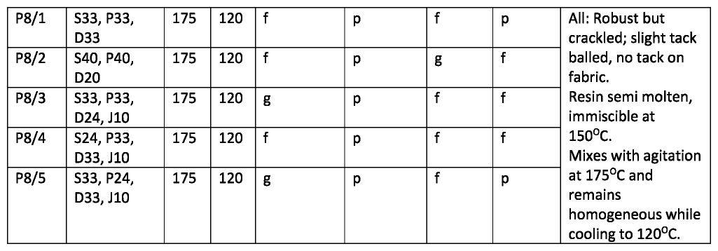

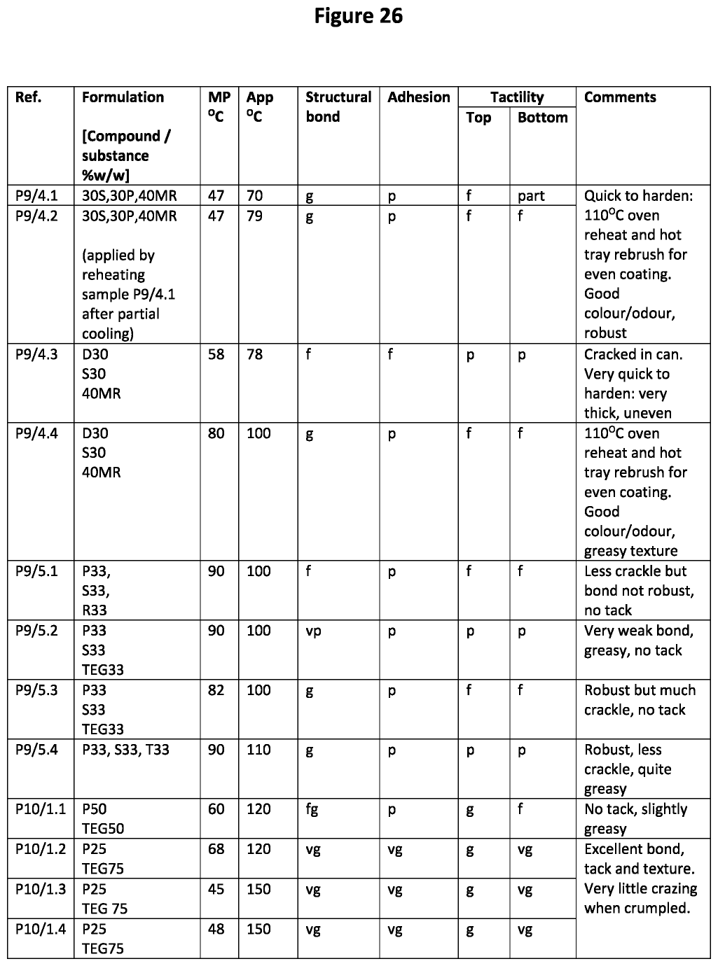

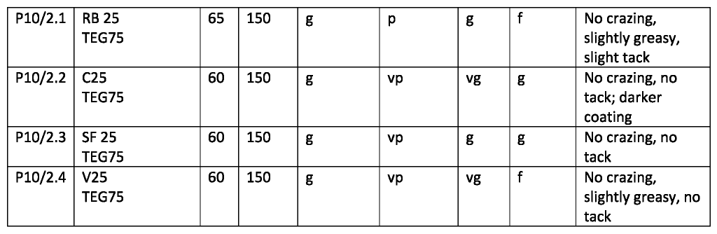

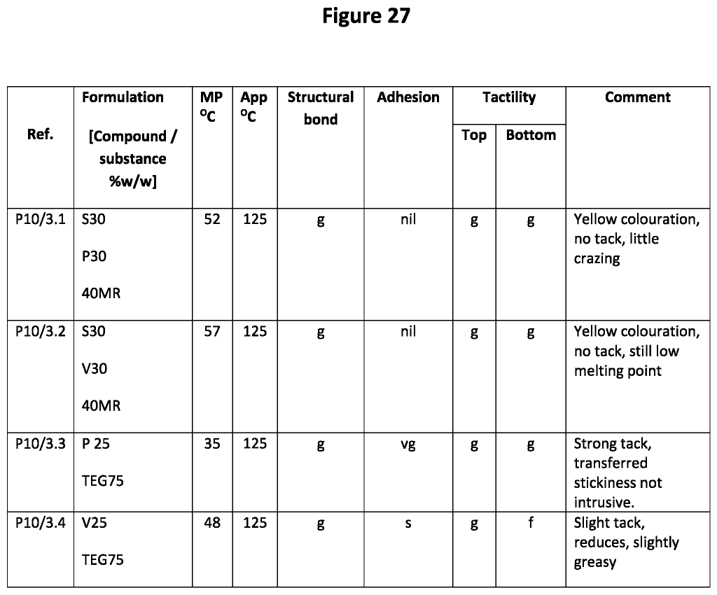

FIGS. 23 to 29 use abbreviations for the compounds used in the formulations trialled. These abbreviations, and the compounds or substances to which they refer, are summarised below:

| | |
|---|---|
| P | Palm wax |
| S | Soy Wax |
| CN | Coconut hydrogenated oil |
| RB | Rice bran wax |
| SF | Sunflower wax |
| J | Jojoba wax |
| B | Bayberry wax |
| C | Candelilla wax |
| R | Pine rosin |
| D | Dammar resin |
| V | Veewax |
| TEG | Triethylene Glycol Rosinate tackifier from |
| | Lawter under the catalogue number TEG |
| | PRLE4014 |
| STA | Fractionated rosin available from Lawter |
| | under the brand name PINEREZ ™ 2210. |
| STB | Fractionated rosin available from Lawter |
| | under the brand name PINEREZ ™ 2402. |
| STC | Fractionated rosin available from Lawter |
| | under the brand name PINEREZ ™ 2314. |

In addition, FIGS. 23 to 29 use abbreviations for various of the test results. These abbreviations, and the meaning of them, are:

| Abbreviation | Meaning |
|---|---|
| F | Fair |
| P | Pass |
| G | Good |
| VG | Very good |
| S | Soft |
| PF | Poor and fair tactility |
| D | Dry |
| S | Soft |
| SG | Soft and greasy |
| DS | Dry and soft |
| DST | Dry, soft and tears |

It should also be understood that the abbreviations may be combined.

By way of clarification, FIGS. 23 to 29 note the formulations trialled using the abbreviations outlined above, and a number to indicate the relative amount (% w/w) of each substance/compound. To assist in better understanding the abbreviations used FIGS. 23 to 29, representative examples are clarified as:
Sample reference P7/3 has a formulation of “S30, P30, R40”. This means the formulation contains 30% w/w of soy wax, 30% w/w palm wax and 40% w/w of pine rosin.
Sample reference P11/7.4 has a formulation of “RB20, S20, STA 60”. This means the formulation contains 20% w/w of rice-bran wax, 20% w/w soy wax and 60% w/w of Fractionated rosin available from Lawter under the brand name PINEREZ™ 2210.
Sample reference P10/3.3 has a formulation noted as “P25, TEG75”. This means the formulation contains 25% w/w of palm wax and 75% Triethylene Glycol Rosinate tackifier from Lawter under the catalogue number TEG PRLE4014.

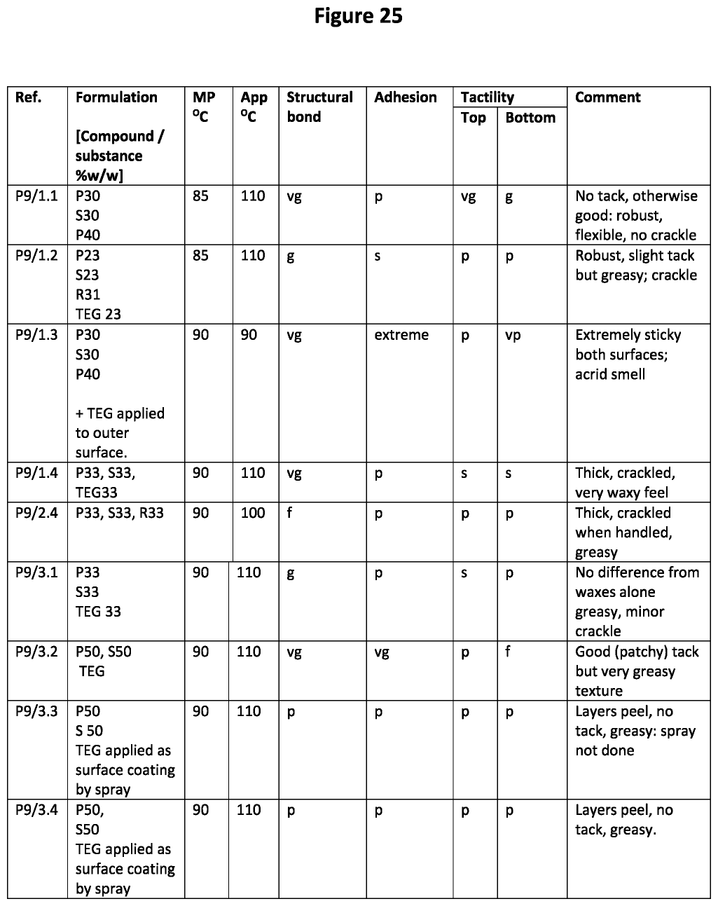

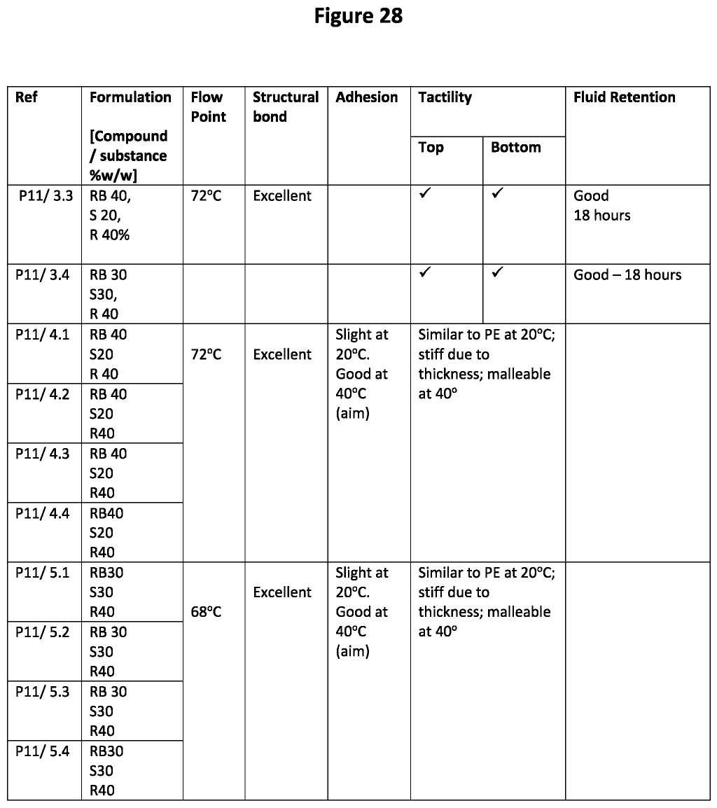

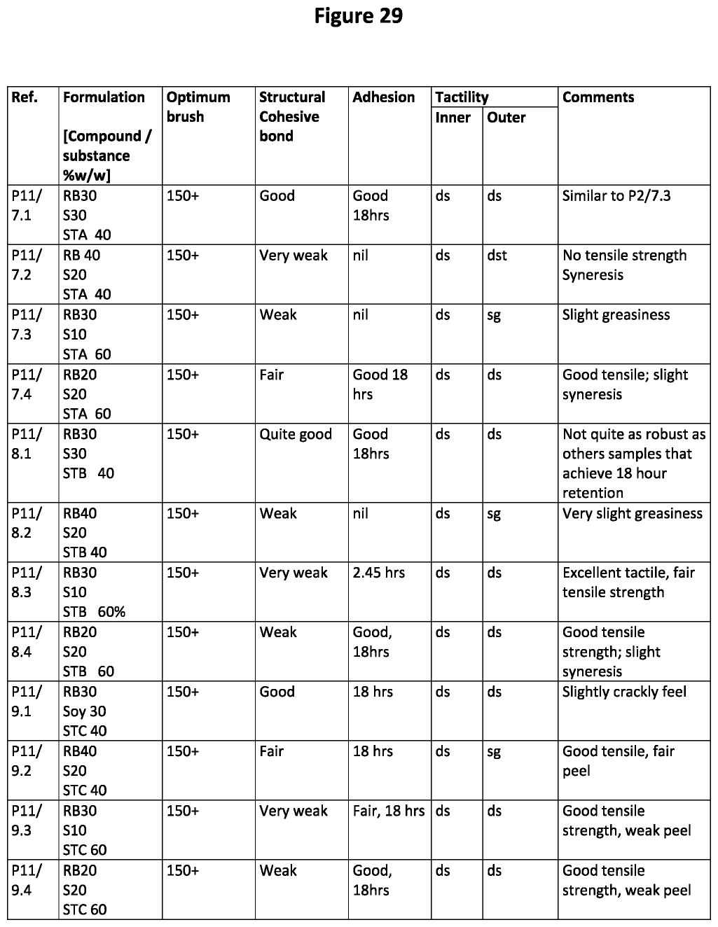

In respect of the trials reported in FIGS. 23 to 29, the inventors note that:
Unless stated otherwise:
   The ingredients were heated to 125° C. to improve ease of mixing and facilitate application to a carrier;
   Melted formulations were maintained at an elevated temperature using a water bath, in the range of 85° C. to 90° C. to facilitate application to a carrier;
   All biodegradable membranes tested included a carrier made from 100% bamboo wadding which included no SAPs, or a commercially available organic cotton sold under the brand Oi Super.
   Flow point of a formulation was assessed after complete melting of wax(es) and tackifier (if used), and as mixture cooled.
   Structural bond was assessed by crumpling the biodegradable membrane in 3 dimensions. The effect on the structure of the membrane was assessed by looking for visible cracks or other damage to the formulation.
   Adhesion was assessed by heating a membrane heated to 38° C. to approximate maximum body-surface temperature, and adhering to a substantially vertical cotton sheet. The length of time the membrane adhered to the sheet was measured as temperature fell to an ambient 20° C.
   Tactility was assessed subjectively by touch to determine the perceived greasiness of the membrane, and whether any tackiness was transferred to the skin, or other uncomfortable sensation experienced.
   Retention tests used a membrane constructed with absorbent fillings as in tests of menstrual pads. Dyed water was added to capacity and weight difference between dry and saturated pads was measured.
The tests reported in FIG. 23 were for a membrane having a sealant formulation as indicated applied to a commercially available table napkin was used as a carrier;
The tests reported in FIG. 25 were for either a membrane having a sealant formulation as indicated applied to a carrier in the form of a commercially available cellulose material sold under the brand name Veocel™ with bamboo wading, as an absorbent
Samples with Ref P11/303 and P11/304 reported in FIG. 28 had a liner extracted form a commercially available sanitary pad, as a carrier.
FIG. 29 reports tests of for optimum temperature for the formulations to be applied to a carrier e.g. by brushing as trialled.

These results suggests that the interactions of the waxes and the stabiliser are unpredictable and variable. However, with appropriate variation of the compounds and proportions in accordance with the present disclosure, it is possible to produce sealant formulation with the desired physical properties.

The skilled addressee would be able to determine how to test the compostability of the formulations disclosed herein, and in particular those exemplified in FIG. 23.

### 7.0 ADVANTAGES

It should be apparent that the present invention may provide a number of advantages compared to the prior art formulations. These may include, but are not limited to:
Being biodegradable in a potentially wider range of conditions e.g. home composting systems or the natural environment.
Being able to degrade even where a product is wrapped upon itself after use. This is compared to current products which are designed to be robustly fluid-proof and therefore, when wrapped inside their own outer coating, may degrade relatively slowly.
Being degradable without any special conditions/sorting. This is compared to current products where if they are not sorted, but pass into general waste, result in a slower rate of biodegradation and means that they do not fully break down while in the upper layers of landfill but become buried deeply, where anaerobic degradation produces methane.
The biodegradable membranes of the present invention do not contain any plastic and therefore do not produce any toxic residues on degradation. This is in contrast to other biodegradable products which contain bioplastics or petrochemical based plastics.
The present invention will degrade naturally without leaving “micro-plastics” behind. Unlike some current products which claim to “biodegrade” but leave microscopic plastic particles in the soil, which do not biodegrade, and may be toxic and harmful to the environment.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

What we claims is:

1. A membrane, wherein the membrane includes
a carrier,
a formulation provided to the carrier, and
a stabiliser.

2. The membrane as claimed in claim 1, wherein the biodegradable membrane is biodegradable in domestic composting conditions as specified in EN 13432, ASTM D6400.

3. The membrane as claimed in either one of claim 1 or 2, wherein the membrane biodegrades within 30 to 60 days.

4. The membrane as claimed in either one of claim 1 or 2, wherein the membrane biodegrades in 18 days or less.

5. The membrane as claimed in any one of claims 1 to 4, wherein the carrier is a structure which comprises a plurality of filaments.

6. The membrane as claimed in claim 5, wherein the structure is formed from a fibrous material.

7. The membrane as claimed in claim 6, wherein the fibrous material is cellulose.

8. The membrane as claimed in any one of claims 1 to 7, wherein the formulation comprises at least one wax.

9. The membrane as claimed in as claimed in any one of claims 1 to 7, wherein the sealant formulation comprises a mixture of at least two waxes.

10. The membrane as claimed in claim 9, wherein the mixture of at least two waxes is a substantially homogenous mixture.

11. The membrane as claimed in either one of claim 9 or 10, wherein the at least two waxes comprises a first wax which has a first hardness and a second wax that has a second hardness, and further wherein the first hardness is relatively hard compared to the second hardness.

12. The membrane as claimed in claim 11, wherein the mixture comprises substantially 30% w/w of the first wax.

13. The membrane as claimed in either one of claim 11 or 12, wherein the mixture comprises substantially 30% w/w of the second wax.

14. The membrane as claimed in any one of claims 11 to 13, wherein at least one of the first wax and the second wax is a natural wax selected from the list of plant waxes, animal waxes, waxes formed by modification of plant waxes or animal waxes, or combinations thereof.

15. The membrane as claimed in any one of claims 11 to 14, wherein at least one of the first wax and the second wax is selected from the list of palm wax, soy wax, bees wax, carnauba wax, rice bran wax, sugarcane wax, sunflower wax, veewax, candelilla wax, rapeseed HO wax, hemp HO wax, Japan wax, Myrica wax, bayberry wax, coconut HO wax, jojoba wax, or combinations thereof.

16. The membrane as claimed in any one of claims 1 to 15, wherein the formulation has a melting point of at least 70° C.

17. The membrane as claimed in claim any one of claims 1 to 16, wherein the formulation has a melting point in the range of substantially 70° C. to substantially 110° C.

18. The membrane as claimed in any one of claims 1 to 17, wherein the stabiliser is admixed into the first wax and the second wax to form a homogenous mixture.

19. The membrane as claimed in any one of claims 1 to 17, wherein the stabiliser is applied to an outer surface of the formulation.

20. The membrane as claimed in any one of claims 1 to 19, wherein the stabiliser is a natural plant substance.

21. The membrane as claimed in any one of claims 1 to 19, wherein the stabiliser is a plant extract or modified plant extract.

22. The membrane as claimed in claim 21, wherein the plant extract is a plant resin or derived from a plant resin.

23. The membrane as claimed in any one of claims 1 to 22, wherein the stabiliser is a food-grade compound.

24. The membrane as claimed in any one of claims 1 to 22, wherein the stabiliser is selected from the list of Pine Rosin or the list of Abietic Acid, Glycerol Ester of Wood Resin, Fumaric Modified Rosin Pentaerythritol Ester, or mixtures thereof.

25. The biodegradable composition as claimed in any one of claims 1 to 24, wherein the stabiliser comprises substantially 40% w/w of the formulation.

26. A formulation for use in manufacturing a membrane, wherein the formulation comprises a mixture of at least two waxes and a stabiliser.

27. The formulation as claimed in claim 26, wherein the mixture of at least two waxes is a substantially homogenous mixture.

28. The formulation as claimed in either one of claim 26 or 27, wherein the at least two waxes a first wax which has a first hardness and a second wax that has a second hardness, and further wherein the first hardness is relatively hard compared to the second hardness.

29. The formulation as claimed in claim 28, wherein the mixture comprises wherein the mixture comprises between substantially 25% to 40% w/w of the first wax, and preferably substantially 30% w/w of the first wax.

30. The formulation as claimed in either one of claim 28 or 29, wherein the mixture comprises between substantially 15-35% w/w of the first wax, and more preferably substantially 30% w/w of the second wax.

31. The formulation as claimed in any one of claims 26 to 30, wherein at least one of the first wax and the second wax is a natural wax selected from the list of plant waxes, animal waxes, waxes formed by modification of plant or animal waxes, or combinations thereof.

32. The formulation as claimed in any one of claims 26 to 30, wherein the first wax is selected from the list of candelilla wax, carnauba wax, palm wax, rice bran wax, sugarcane wax, sunflower wax, modifications and derivatives of the foregoing, and combinations thereof.

33. The formulation as claimed in any one of claims 26 to 32, wherein the second wax is selected from the list of bayberry wax, coconut HO wax, hemp HO wax, Japan wax, jojoba wax, Myrica wax, rapeseed HO wax, soy wax, veewax, modifications and derivatives of the foregoing, and combinations thereof.

34. The formulation as claimed in any one of claims 1 to 33, wherein formulation has a melting point of at least 70° C.

35. The formulation as claimed in any one of claims 26 to 34, wherein the sealant formulation has a melting point in the range of substantially 70° C. to substantially 110° C.

36. The formulation as claimed in any one of claims 26 to 35, wherein the stabiliser is a tackifier.

37. The formulation as claimed in claim 36, wherein the stabiliser is admixed with the first wax and the second wax to form a homogenous mixture.

38. The formulation as claimed in either one of claims 35 to 37, wherein the stabiliser is a natural plant substance.

39. The formulation as claimed in any one of claims 35 to 38, wherein the stabiliser is a plant extract.

40. The formulation as claimed in claim 39, wherein the plant extract is a plant resin.

41. The formulation as claimed in any one of claims 26 to 40, wherein the stabiliser is a food-grade compound.

42. The formulation as claimed in any one of claims 26 to 41, wherein the stabiliser is selected from the list of Pine Rosin or the list of Abietic Acid, Glycerol Ester of Wood Resin, Fumaric Modified Rosin Pentaerythritol Ester, or mixtures thereof.

43. The formulation as claimed in any one of claims 26 to 42, wherein the stabiliser comprises substantially 40% w/w to 60% w/w of the sealant formulation.

44. A method of manufacturing a membrane, the method including the steps of:
heating a formulation as claimed in any one of claims 26 to 43 to form a melted mixture, and
applying the melted mixture to a carrier.
